(19) United States
(12) Patent Application Publication
(10) Pub. No.: US 2024/0085412 A1
(43) Pub. Date: Mar. 14, 2024

(54) Vector Transmitted Infectious Disease Assay

(71) Applicant: Tick Off, LLC, Rockingham, VA (US)

(72) Inventors: William John HALL, Harrisonburg, VA (US); Mark LIPSKY, Encinitas, CA (US)

(21) Appl. No.: 18/232,995

(22) Filed: Aug. 11, 2023

Related U.S. Application Data
(60) Provisional application No. 63/397,734, filed on Aug. 12, 2022.

(51) Int. Cl. G01N 33/569; G01N 1/28; G01N 33/543

(52) U.S. Cl. CPC G01N 33/56911; G01N 1/286; G01N 33/54388; G01N 2001/2866; G01N 2333/20; G01N 2469/10

(57) ABSTRACT

An assay test kit for testing an insect for an infectious disease comprises an insect macerator and a lateral flow assay test strip. The kit may further comprise a liquid carrier, a filter, an assay sample dispenser, and an insect capture tool.

### BACKGROUND

Lyme disease is the most common vector-borne disease in the United States, spread by ticks infected with the Borrelia burgdorferi bacteria. Initial symptoms include rash, fever, and fatigue, and if left untreated can produce severe headaches, facial paralysis, irregular heartbeat, and inflammation of the brain and spinal cord. Diagnosis of Lyme disease is generally performed by blood testing of the subject for antibodies to the disease bacteria.

Risk of Lyme disease transmission is dependent on the length of time a tick is attached to the host and also the likelihood that the tick itself is a carrier of the disease bacteria. This likelihood can range from less than 1% to greater than 50% over time and in different geographic regions. Therefore, methods of testing ticks themselves for the Borrelia burgdorferi bacteria can be useful for those living in areas where infected ticks may be common to help manage Lyme disease risk, take relevant precautions, and if infected or suspected to be infected, obtain prompt treatment.

Currently, testing ticks for the Lyme disease bacteria requires capturing the tick of interest and mailing it to a laboratory where the tick is actually tested. This takes many days and is expensive.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

In one implementation a kit for testing an insect for an infectious agent is provided. The kit comprises an insect macerator suitable for use in a non-laboratory environment. The kit may further comprise a lateral flow assay test strip. In various implementations, the kit may comprise one or more of a lateral flow assay test strip reader, liquid carrier, one or more filters, an assay sample dispenser, and an insect capture tool.

In another implementation, an insect processing tool for use in preparing a liquid sample for a lateral flow assay comprises a first component comprising a well having a floor, wherein the floor is configured for supporting a live insect on the floor, and wherein the walls of the well are configured to inhibit the live insect from climbing out of the well. The tool further comprises a second component comprising a post configured for placement within the well. The first component and the second component are threadably engageable to crush an insect between the post and the floor of the well.

In another implementation, a method of testing an insect for a vector transmitted infectious disease comprises outside of a laboratory environment, crushing the insect and mixing crushed portions of the insect with a liquid carrier, and outside of a laboratory environment, applying the liquid carrier that has been mixed with the crushed portions of the insect to a lateral flow assay strip.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1 is a block diagram illustrating possible components of an assay test kit according to some embodiments.

FIG. 2 shows exemplary assay test kit components of insect macerator and lateral flow assay test strip in accordance with some embodiments.

FIG. 3 shows exemplary lateral flow assay test strips with assay test strip readers.

FIG. 4A is a perspective view of an insect macerator prior to threaded coupling according to some embodiments;

FIG. 4B is a cross sectional view of the insect macerator of FIG. 4A;

FIG. 5A is a perspective view of the insect macerator of FIG. 4A after threaded coupling according to some embodiments;

FIG. 5B is a cross sectional view of the insect macerator of FIG. 5A;

FIG. 6 is a cross sectional view of another embodiment of an insect macerator;

FIG. 7 shows components of an assay test kit including the insect macerator of FIG. 6 according to some embodiments;

FIG. 8 shows the assay test kit components of FIG. 7 in use prior to mixing crushed portions of an insect with a liquid carrier;

FIG. 9 shows the assay test kit components of FIG. 7 in use after mixing crushed portions of an insect with a liquid carrier;

FIG. 10 shows the assay test kit components of FIG. 7 after separating the sample reservoir from the insect macerator;

FIG. 11 shows other exemplary test kit components of insect macerator, filter, and assay sample dispenser;

FIGS. 12A through 12G illustrate an exemplary process for testing an insect for an infectious agent in accordance with some embodiments.

FIG. 13 illustrates exemplary insect capture tools that may be part of an assay test kit in accordance with some embodiments.

FIG. 14A shows an exemplary assay test kit according to some embodiments;

FIG. 14B shows another exemplary assay test kit according to some embodiments;

FIG. 14C shows another exemplary assay test kit according to some embodiments.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

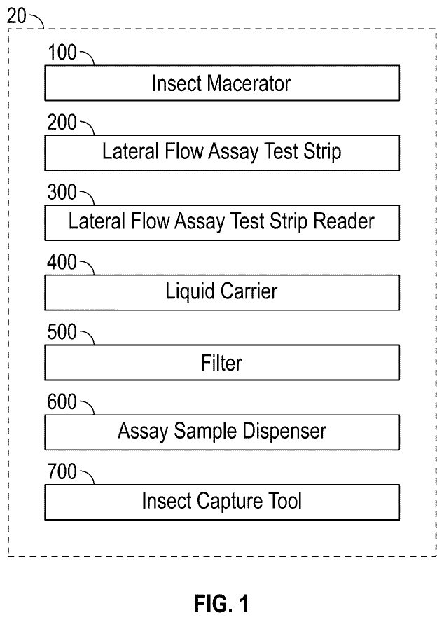

FIG. 1 is a block diagram of an assay test kit 20 for testing an insect for the presence of an infectious agent. One suitable application of the apparatuses and methods described herein is testing ticks for the presence of Lyme disease bacteria such as the Borrelia burgdorferi bacteria. However, the apparatuses, methods, principles, and inventive concepts described herein are not so limited, and may be applied to a wide variety of insects, animals, plants, or any disease vector.

Referring now to FIG. 1, the test kit 20 may include an insect macerator 100 and a lateral flow assay test strip. In some embodiments, the test kit is intended to be used in a non-laboratory environment such as a home, office, or other location or facility that is not equipped with laboratory equipment for performing chemical or biological assays. Thus, the facility for preparing a suitable sample for performing an assay (e.g. an insect macerator 100) and the device for performing the assay itself (e.g. a lateral flow assay test strip 200) are both provided with the kit. In some embodiments, as described further below, the lateral flow assay test strip has a visually detectable result output. In some embodiments, the kit may include a lateral flow assay test strip reader that uses optical or other means to read the test result from the lateral flow assay test strip and provide the user with a result such as an LED light output or digital YES/NO character display. In some implementations, components of the kit may be shipped or otherwise delivered to a user in different packages. For example, an insect macerator 100 may be re-usable and shipped by itself, and assay test strips may be shipped separately such that the kit is assembled by the user from components received separately by the user. In other implementations, all the components may be shipped together in one package. In this implementation, all the components may be single use, or some of the components may be re-usable with the user obtaining additional assay test strips separately as needed for performing multiple assays.

Other optional components of the assay test kit may include a liquid carrier 400, a filter 500, an assay sample dispenser 600, and an insect capture tool 700. Examples of each of these items are presented below.

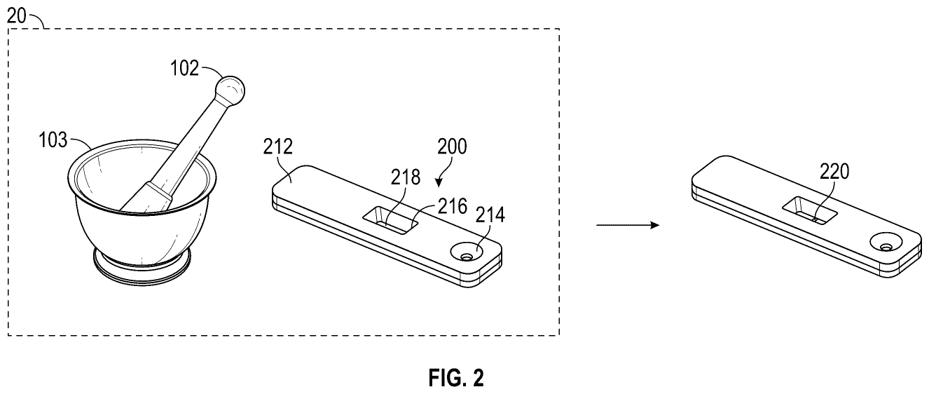

FIG. 2 illustrates a test kit 20 with an insect macerator comprising a mortar 130 and pestle 120. One or both of the mortar 130 and pestle 120 may be made of metal such as stainless steel or may be a polymer. If polymer, it is preferable a relatively hard polymer such as glass reinforced nylon or polycarbonate. Glass or ceramic are also suitable. Also provided is a lateral flow assay test strip 200. In this implementation, the lateral flow assay test strip includes the strip itself 218 enclosed within a housing 212. A sample application port 214 is provided in the housing and a test result region of the test strip 218 is viewable through a window 216 in the housing 210. If the test is positive, the test region of the test strip 218 becomes colored or darkened, typically forming a line 220 viewable in the window 216 and/or electronically readable by electronic test strip reading circuitry (not shown in FIG. 2). The detection functionality of the test strip itself may be immunological, such as an antibody sandwich assay of the type described in U.S. Pat. No. 10,823,726 entitled Bed Bugs Detection Device, the entire content of which is hereby incorporated by reference in its entirety. If the device is configured for testing for Lyme disease, at least some of the antibodies provided on the test strip may be configured to specifically bind to proteins found in the bacteria that cause Lyme disease, such as the Borrelia burgdorferi bacteria.

In the implementation of FIG. 2, the insect such as a tick may be placed in the mortar 130 with a small amount of liquid carrier and crushed with the pestle 120, which may have a roughened bottom surface, or be scored, knurled, or include projections or other features for thoroughly macerating the insect in the bottom of the mortar 130. Some or all of the liquid in the bottom of the mortar following the maceration of the insect is then poured or otherwise applied to the sample application port 214 of the lateral flow assay test strip.

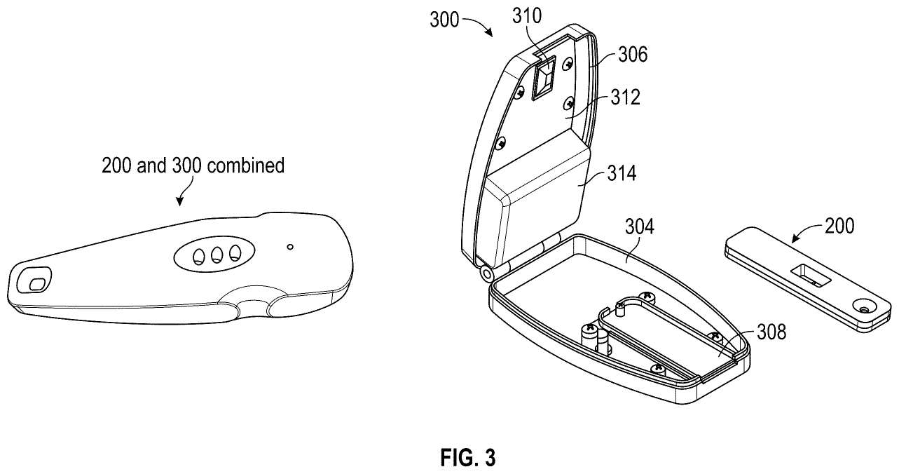

FIG. 3 shows example lateral flow assay test strip readers that may be provided as part of the kit 20 in some embodiments. In the embodiment on the left in FIG. 3, the test strip and reader electronics are enclosed together in a common housing, forming a disposable combination test strip plus reader electronics. In the embodiment on the right in FIG. 3, the reader 300 is a separate component that accepts a test strip 200 for reading. The reader 300 on the right is serially re-usable with multiple test strips 200. In this embodiment, the reader 300 may be of a clamshell type design with an upper enclosure 306 and a lower enclosure 304. One or more LED and/or photodetectors 310 may be provided as part of electronics 312 and a battery pack 314 in the top enclosure 306. The bottom enclosure may include a location 308 for placing the assay test strip 200. These embodiments are described in more detail in U.S. Patent Application Publication 2022/0214284 entitled Apparatus and Methods for Assaying a Liquid Sample, the content of which is incorporated herein by reference in its entirety.

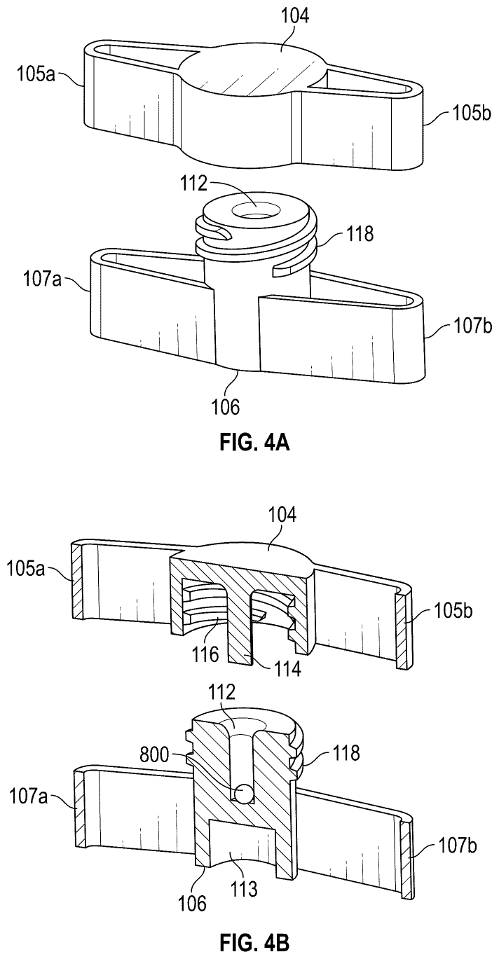

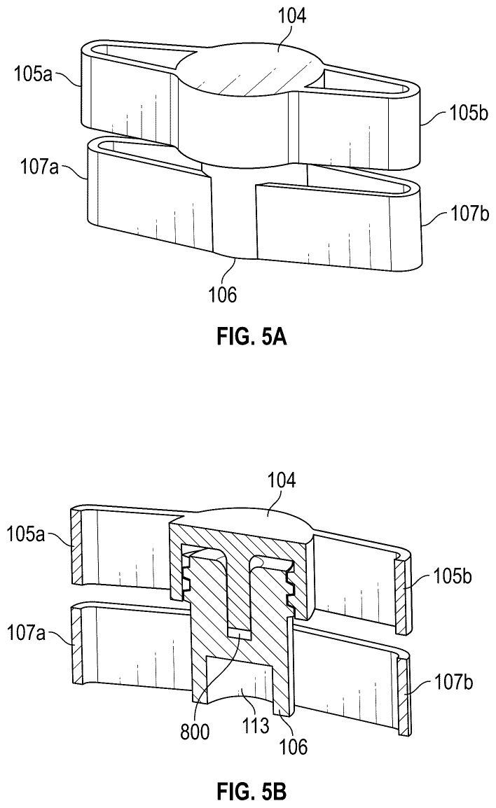

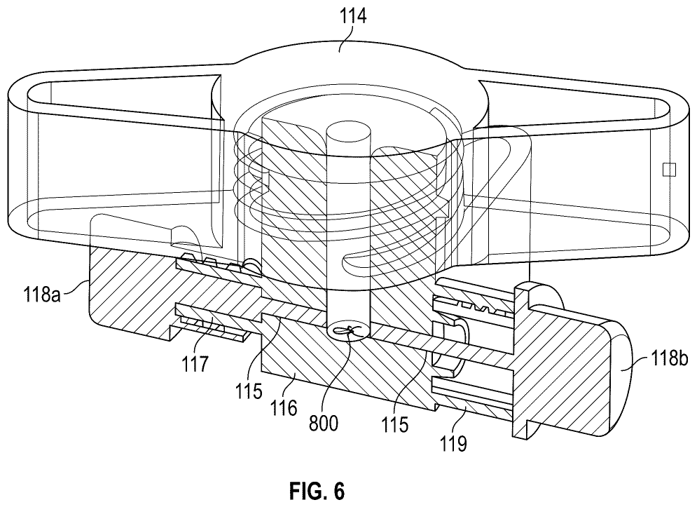

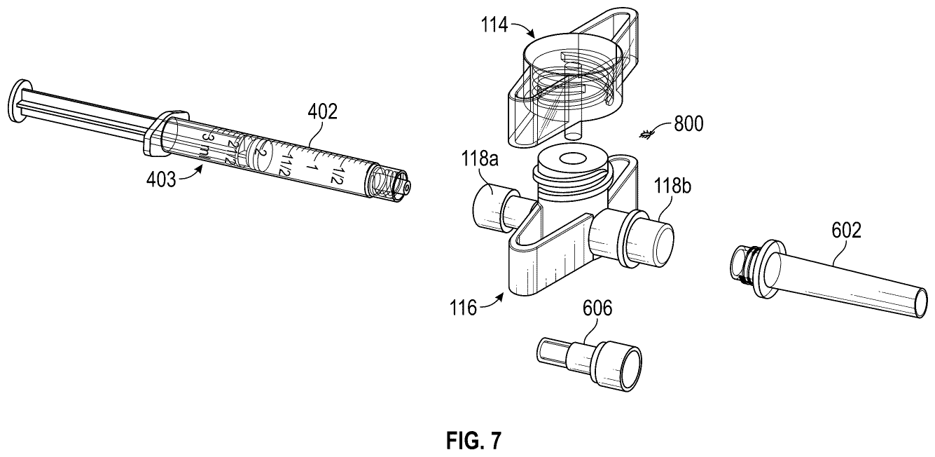

FIGS. 4A, 4B, 5A, and 5B show a first embodiment of an insect macerator for use in a non-laboratory environment. This embodiment is functionally similar to a simple mortar and pestle such as shown in FIG. 2 but may be more user friendly in a non-laboratory environment.

FIG. 4A is a perspective view of the insect macerator and FIG. 4B is a cross sectional view of the insect macerator of FIG. 4B. In this embodiment, the incest macerator comprises a first component 106 and a second component 104. In FIGS. 4A and 4B the two components 104, 106 are uncoupled from each other. The first component 106 comprises a well 112 configured for holding an insect 800 such as a tick. The well 112 has a floor on which the insect 800 is supported. The walls of the well are configured such that a live insect cannot crawl up the sides an escape, e.g. the walls are steep, advantageously vertical, and the depth of the well compared to the size of the insect is sufficient that the insect is far below the well opening when the insect is located on the floor of the well 112. The second component 104 comprises a post 114 configured to fit into the well 112. The fit between the post 114 and the well 112 should allow easy sliding of the post 114 into the well 112 but not provide so much space around the sides of the post for the insect to slip past or into when the post 114 is in the well 112. For example, the widest longitudinal cross-sectional area of the post 114 may be at least 50% of the narrowest cross-sectional area of the well 112. The longitudinal cross-sectional shapes of the well 112 and the post 114 need not be constant along their lengths and also need not be the same. The diameter of the well 112 may, for example, be about 5-10 mm for a device suitable to be used for ticks, with the diameter of the post 114 being 0.1-1.5 mm smaller. The depth of the well may, for example, be about 10-15 mm

The first component 106 and the second component 104 may be threadably engageable. In this implementation, the first component 106 is provided with external threads 118 around the well 112 and the second component 104 is provided with internal threads 116 around the post 114. The first component 106 may comprise flanges 107a and 107b forming handles. The second component 104 may comprise flanges 105a and 105b also forming handles.

Referring now to FIGS. 5A and 5B, the insect macerator of FIGS. 4A and 4B is shown with the threads 116 and 118 engaged with the first component 106 and the second component 104 screwed together sufficiently to crush the insect 800 between the floor of the well 112 and the end of the post 114. This can be done manually by a user by threading the components 104, 106 until the insect 800 is crushed. When fully engaged, the post 114 may come into contact or near contact with the floor of the well 112 to ensure that an insect will be crushed between the bottom of the post 114 and the floor of the well 112.

After the insect 800 is crushed, a liquid carrier may be deposited in the well 112. This liquid carrier may be a buffer solution provided as part of a kit or may be plain tap water provided by the user. The crushed insect 800 and liquid carrier may be mixed with a tool such as toothpick or a tool provided in the kit. Additionally or alternatively, the liquid carrier and crushed insect 800 may be mixed by partially threading the second component 104 back onto the first component 106 and manually agitating the incest macerator. Instead of re-threading the second component 104 onto the first component 106 for this purpose, a separate cap (not shown) may be provided as part of the kit. The mixture may be allowed to set for a time to allow biological chemicals from the crushed tick to solubilize in the liquid carrier. After mixing and incubating, the liquid carrier may be poured onto a lateral flow assay strip such as shown in FIGS. 2 and 3 for performing the assay.

In some implementations, another well 113 may be provided on the underside of the first component. This well may be capped and may contain the liquid carrier for the kit. In this implementation, it is possible to make the wall of the first component between the floor of the well and the chamber 113 very thin such that when the insect is crushed, the post can push through that wall and push the crushed insect into the capped well 113 containing the liquid carrier. The insect macerator can then be agitated, the mixture incubated, and then the cap on the chamber 113 can be removed such that the liquid carrier mixed with the crushed insect can be poured onto a lateral flow assay test strip as described above.

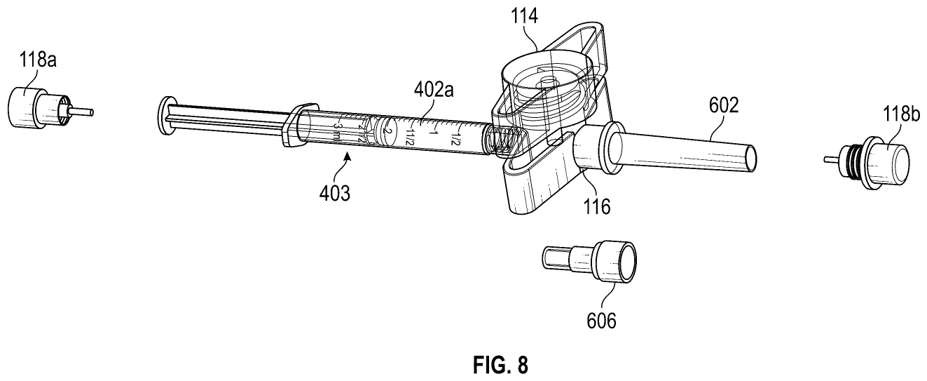

FIG. 6 shows another embodiment of an insect macerator. This embodiment is similar to that shown in FIGS. 4A through 5B except in this embodiment a liquid flow channel 115 is provided that intersects the well near the floor of the well. This liquid flow channel 115 may have an entry port 117 on one side of the first component 116 and an exit port 119 on the other side of the first component 116. The entry port 117 and exit port 119 may be closed with caps 118a and 118b respectively while the insect is crushed against the floor of the well as described above with respect to the embodiment of FIGS. 4A through 5B. As described further below, this liquid flow channel 115 can be used to mix the crushed insect 800 with a liquid carrier.

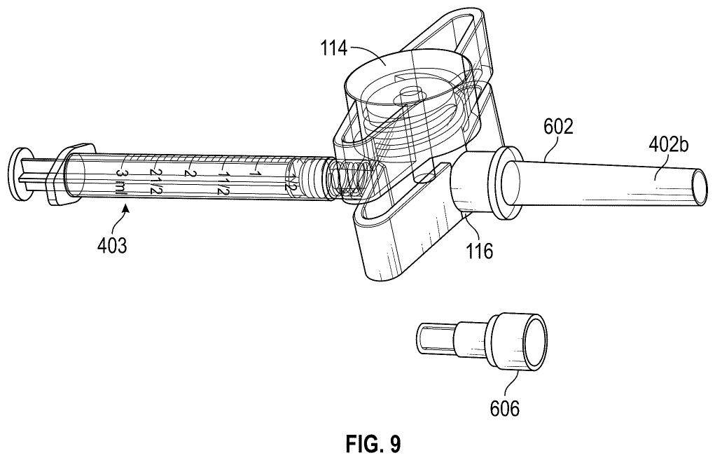

Referring now to FIG. 7, in this embodiment, the kit may further comprise a syringe 403 containing a liquid carrier 402a and a sample reservoir 602 with a cap 606. The sample reservoir can be used as an assay sample dispenser to place the sample on the assay test strip. In use, the insect 800 is first placed into the well of the first component 116 and crushed against the floor of the well with the post of the second component 114 as is also described above with respect to the embodiment of FIGS. 4A through 5B. For this portion of the procedure, the caps 118a and 118b remain in place.

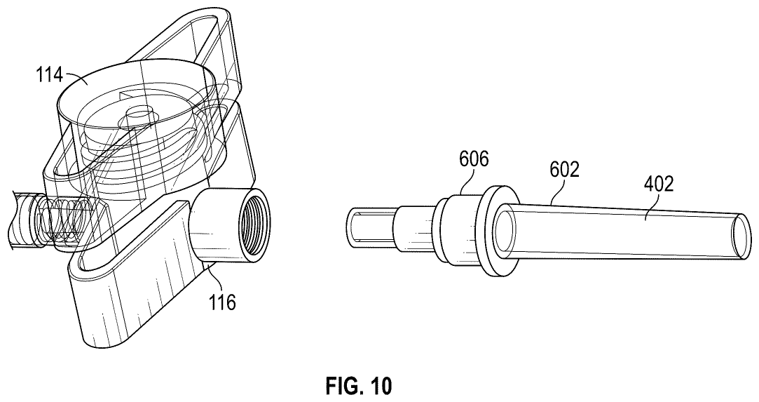

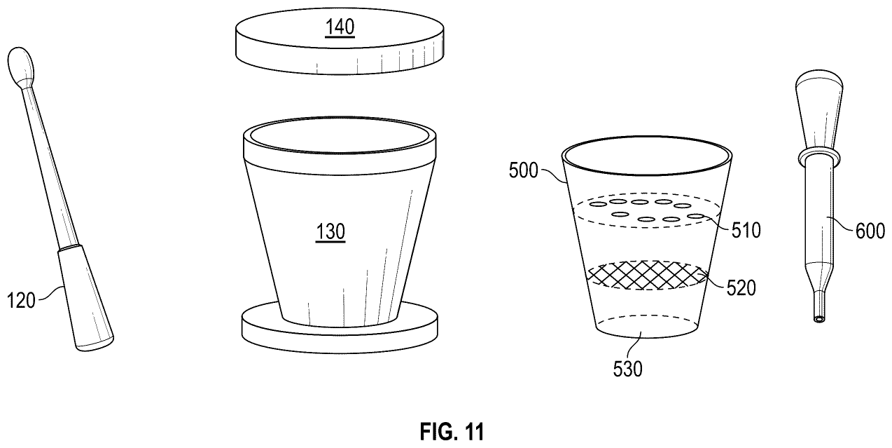

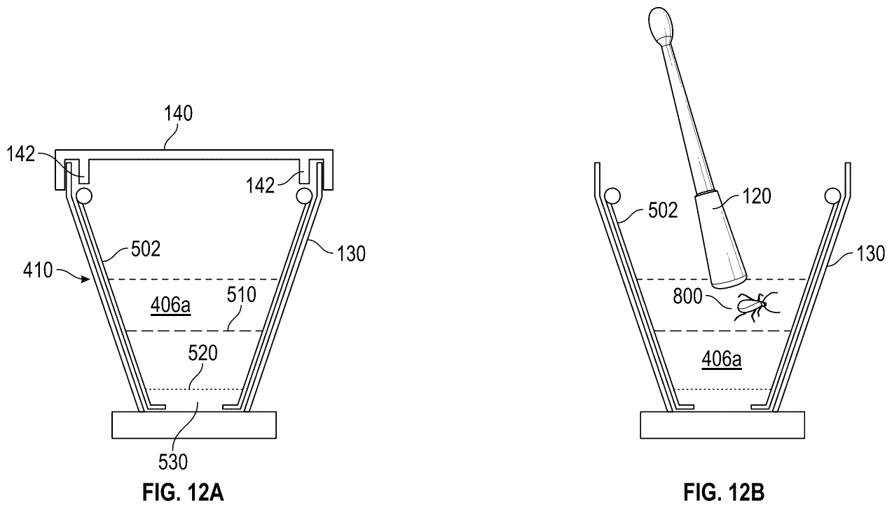

Referring now to FIG. 8, after the first component 116 and second component 114 are threadably engaged and the insect 800 is crushed, the syringe 403 can be attached to the entry port 117 of the fluid channel 115 and the sample holding reservoir 620 can be attached to the exit port 119 of the fluid channel 115. As shown in FIG. 9, syringe 403 plunger can be pressed down to expel the liquid carrier 402a from the barrel of the syringe, down the fluid flow channel 115, over and through the crushed insect in the well, and into the sample reservoir 602. As shown in FIG. 10, the sample reservoir 602 can be removed and capped with the cap 606. After this, the liquid carrier mixed with the crushed insect 402b can be deposited onto the assay test strip 200 to generate the assay results. In some embodiments, two syringes can be provided, one of which contains the liquid carrier 402 and the other of which is empty. After crushing the insect, the syringe 403 can be placed on the entry port 117 as above, and the second syringe, with plunger down, can be attached to the exit port 119. The entry and exit ports can have the same connection format in this embodiment. The liquid carrier 402 can be pushed from the first syringe 402 through the flow channel 115, over and through the bottom of the well with the crushed insect and into the other syringe at the exit port 119, pushing its plunger out as it fills with liquid carrier 402. This could then be repeated in the other direction, passing the liquid carrier back over and through the crushed insect. This may be repeated back and forth to provide several passes over and through the crushed insect at the bottom of the well to help thoroughly solubilize the biochemical substances from the crushed insect into the liquid carrier. Afterwards, either syringe could be used as the assay sample dispenser 600 to place the sample on the assay test strip.

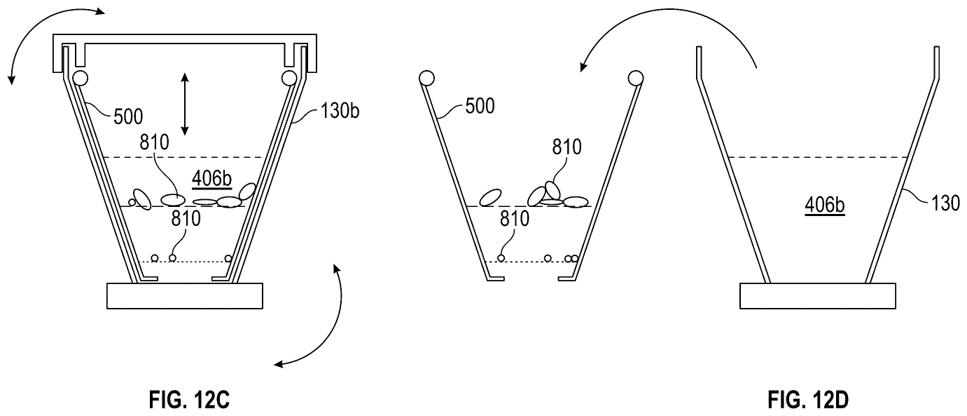

FIG. 11 illustrates insect macerator 100, filter, 500, and sample dispenser 600 components of another exemplary assay test kit in accordance with some embodiments. In this implementation, the macerator includes the mortar 130 and pestle 120 described above, further including a lid or cover 140. As described above, one or both of the mortar 130 and pestle 120 may be made of metal such as stainless steel or may be a polymer. If polymer, it is preferable a relatively hard polymer such as glass reinforced nylon or polycarbonate. Glass or ceramic are also suitable. The lid 140 may also be made of the same materials, may be screw on to the mortar 130, or may be a softer flexible plastic or rubber which can be press file over the rim of the mortar to provide a substantially liquid tight seal. In this implementation, the mortar 130 has inwardly sloping sides and is shaped as a truncated downwardly pointing cone.

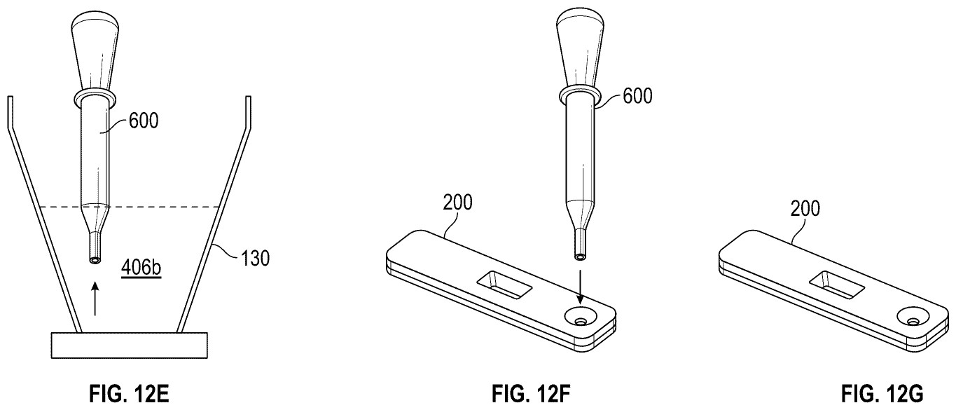

Also provided is a filter insert 500 which has inwardly sloping sides that substantially match the inwardly sloping sides of the mortar 130, allowing the filter insert 500 to rest inside the mortar 130 as will be described in further detail below with reference to FIGS. 12A through 12D. The filter insert 500 includes at least one filtering plane 510 that segments the inside of the filter insert into upper and lower compartments. In the implementation of FIG. 11, there are two filtering planes 510, 520 having different coarseness or porosity for filtering. The filter insert may also be metal or polymer similar to the mortar 130 and may have solid sidewalls although in FIG. 4 the sidewalls are shown transparent for convenience in illustrating the filtering planes 510 and 520 inside. The bottom 530 of the filter insert 500 may be substantially open.

The assay sample dispenser 600 may be an eyedropper style pipette with a hand actuatable bulb at the top for aspirating and dispensing liquids into and out of the column reservoir at the bottom. Such pipettes are inexpensive single piece plastic that are commercially available for a wide variety of uses.

FIGS. 12A through 12G illustrate the performance of an assay process using the apparatus illustrated in FIG. 4. In FIG. 5A, a mortar 130 is shown with a filter insert 500 resting inside. The filter insert has a first filtering plane 510 which segments the internal volume of the filter insert 500 into upper and lower portions. The filtering plane 510 may be metal or hard plastic and may contain through holes forming a sieve of a first filtering porosity. The filter insert 500 may also comprise a second filtering plane 520 beneath the first filtering plane 510. The second filtering plane may be a polymer or metal fine mesh or screen or even a glass frit providing a finer filtering capability than the first filtering plane 510. The mortar 130 is filled to a fill level 410 with a liquid carrier 406. The fill level 410 may be somewhat above the level of the first filtering plane 510. The liquid carrier 400 may be simply water, may be a buffer, and may include agents for assisting the solubilization of bacteria, viruses, or components thereof that are desired to be detected. The mortar 130 is covered with the lid 140 in a substantially liquid tight manner. The underside of the lid 140 may include protrusions 142 or other features to hold the filter insert 500 in place against the sides and bottom of the inside of the mortar 130 when the lid 140 is attached to the mortar 130. The apparatus shown in FIG. 5A may be delivered to a user as part of an assay test kit in the configuration shown, that is, filter pre-installed, mortar pre-filled with liquid, and sealed with the lid 140. In other alternatives, the liquid carrier is added to the mortar 130 by the user, for example plain water or a carrier provided in a separate container with the assay test kit.

To use the assembly of FIG. 12A, the user removes the lid 140, and places an insect 800 such as a tick into the mortar 130 and the liquid carrier 400 therein. As shown in FIG. 12B, using the pestle 120, the user crushes and macerates the tick against the first filter plane 510. The filter plane 510 is made of a polymer or metal material and has a thickness sufficient to withstand the insect macerating process without substantial mechanical deformation or damage. Like the bottom of the pestle 120, the top of the filter plane 510 may be knurled, scored, or provided with protrusions or other features to facilitate thorough maceration of the insect. The finer mesh filter 520 need not be constructed to withstand the forces of maceration and can consequently be made with fine mesh features without risk of damage during use.

As shown in FIG. 12C, the lid 140 may be placed back on the mortar 130, and the assembly may be shaken, turned over, and otherwise agitated by the user to fully release the desired bacteria and/or virus components from the macerated insect material into the liquid carrier 406. Broken pieces of insect exoskeleton or other insoluble insect parts 810 will remain confined to the inside of the filter insert by filter planes 510 and 520. The assembly of FIG. 12C may also be allowed to rest and incubate for a desired amount of time.

Referring now to FIG. 12D, the lid 140 may be removed and the filter insert 500 lifted out of the mortar 130. The filter planes 510 and 520 will retain and remove the insoluble insect debris, leaving the liquid carrier 406 with dissolved and solubilized insect components, including any bacteria or viruses to be detected, behind in the mortar 130.

In FIGS. 12E and 12F, the assay sample dispenser 600 is used to aspirate some or all of the liquid carrier 400 from the mortar 130 and dispense it onto the sample receiving port of the lateral flow assay test strip 200. In FIG. 12G, the result is read, either visually or electronically.

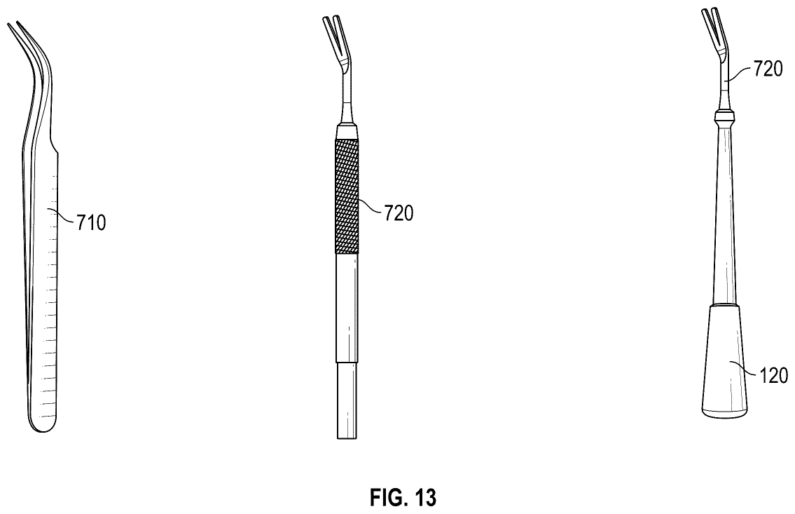

FIG. 13 illustrates insect capture tools 700 that may be provided with the assay test kit in some embodiments. An insect capture tool may be simply tweezers 710 as shown on the left. In the center, a tool 720 specifically designed for removing ticks from the skin is shown. On the right, a combined tick remover 720 and pestle 120 is illustrated. Such a combination of macerator components and insect capture components can be achieved in the embodiments of FIGS. 4A to 10 as well. For example, a forked tick removal tool could be integrally molded to extend out from the outer end of the handle flange 105b of the second component 104 of the embodiment illustrated in FIGS. 4A and 4B. The user could collect a tick with this integral tool on component 104 and drop it into the well 112 of the first component 106.

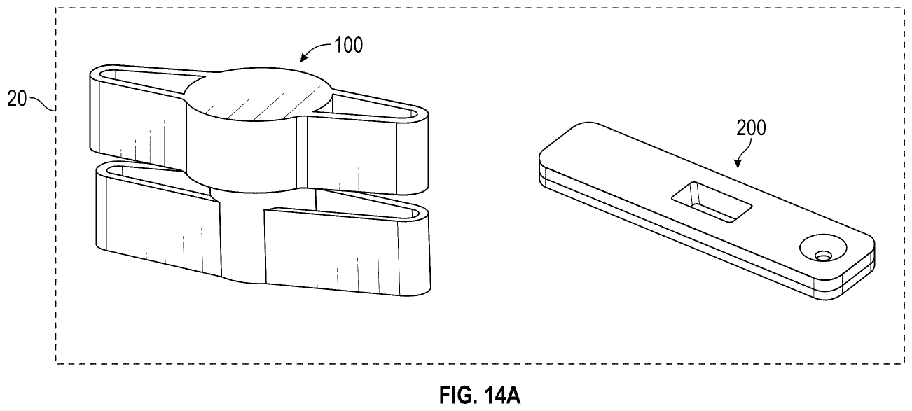

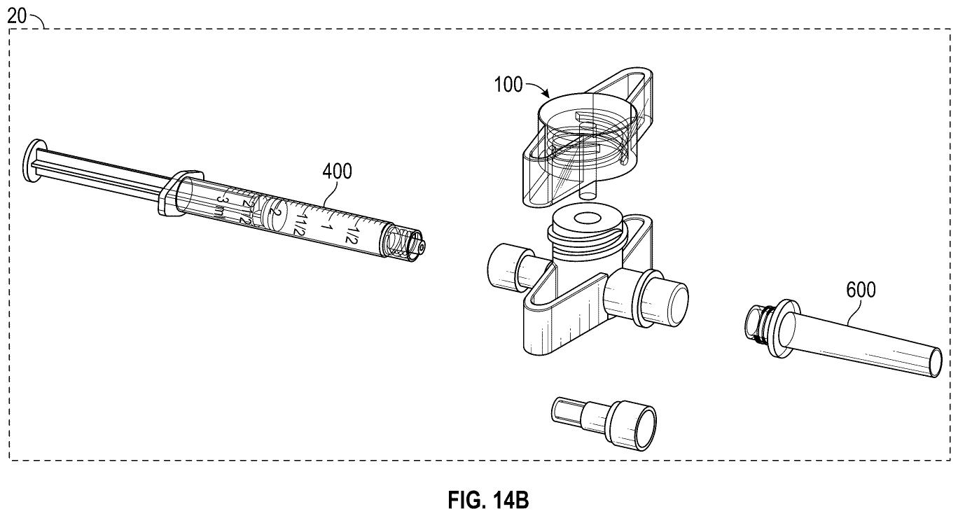

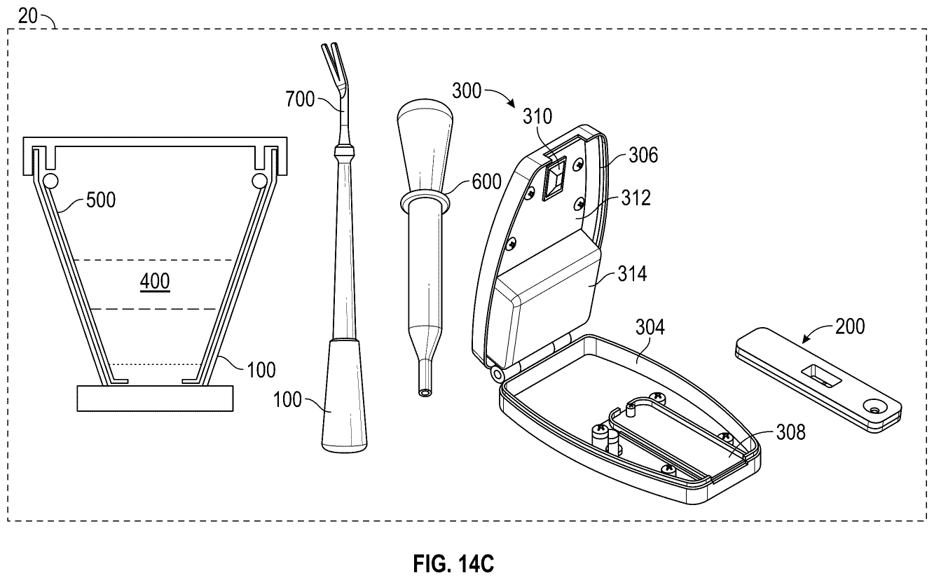

FIG. 14A illustrates a kit for performing an assay comprising an insect macerator 100 and a lateral flow assay test strip 200. In this embodiment, the insect macerator is as described above with reference to FIGS. 4A to 5B. In this embodiment, the kit includes only items 100 and 200 from FIG. 1. As also noted above, these items could be delivered separately to the user, who then assembles the kit for performing the assay. FIG. 14B illustrates a kit 20 containing an insect macerator 100, liquid carrier 400, and an assay sample dispenser 600. FIG. 14C shows an assay test kit in accordance with some embodiments that includes all of the components of the assay test kit 20 of FIG. 1. As noted above, many of the items in the assay test kit 20 of FIG. 14C are optional. Also, the disposability or reusability of the components can differ in different embodiments. In some embodiments, every component shown in FIG. 14C and listed in FIG. 1 is single use disposable. In some embodiments, the reader 300 is reusable but the other components are single use disposable. In some embodiments, the mortar 130, pestle 120, lid 140, and filter insert 500 are reusable, and may be dishwasher safe for example. In one implementation, no reader is provided, a plurality of human readable lateral flow assay test strips are provided, and the mortar, pestle, and filter insert are reusable for the plurality of lateral flow assay test strips provided and are then disposable. In these embodiments, the liquid carrier 400 may be water provided by the user or a container of liquid carrier may be provided for performing the plurality of tests corresponding to the plurality of lateral flow assay test strips that are provided. Different combinations other than those specifically listed are also possible.

The insect macerator 100 can take a number of other forms as well for facilitating the release of appropriate antigens from an insect into a liquid carrier for application to an assay test strip. For example, the insect macerator may comprise a bag or pouch with flexible side walls. The insect can be placed in the bag or pouch and the user can put pressure on the insect from the outside either with bare fingers or with a tool similar to pliers that may or may not be provided as part of the kit to crush the insect. The inside of the surfaces of the bag or pouch walls could have bumps or small spikes to assist the crushing process. The pouch could already contain liquid carrier before the insect is placed inside, liquid carrier could be added after the insect is crushed, or the bag or pouch (or a portion thereof) could be placed into a separate container of liquid carrier after the tick is crushed.

General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

What is claimed is:

1. A kit for testing an insect for an infectious agent, the kit comprising:
an insect macerator; and
a lateral flow assay test strip.

2. The kit of claim 1, comprising a liquid carrier.

3. The kit of claim 2, wherein the liquid carrier is contained in a syringe.

4. The kit of claim 1, comprising an insect capture tool.

5. The kit of claim 1, comprising an assay sample dispenser.

6. The kit of claim 1, comprising a lateral flow assay test strip reader.

7. The kit of claim 6, wherein the lateral flow assay test strip and the lateral flow assay test strip reader are contained within a common housing.

8. The kit of claim 6, wherein the lateral flow assay test strip and the lateral flow assay test strip reader are separate components.

9. The kit of claim 8, wherein the lateral flow assay test strip reader is configured to be used serially with a plurality of lateral flow assay test strips.

10. The kit of claim 1, wherein the lateral flow assay test strip comprises an antibody that binds to a protein associated with the Borrelia burgdorferi bacteria.

11. The kit of claim 1, comprising a filter.

12. The kit of claim 11, wherein the filter is configured to engage with or be coupled to at least one component of the insect macerator.

13. The kit of claim 1, wherein the insect macerator comprises first and second components configure to crush an insect therebetween.

14. The kit of claim 13, wherein the first and second components are threadably engageable.

15. An insect processing tool for use in preparing a liquid sample for a lateral flow assay, the insect processing tool comprising:
a first component comprising a well having a floor, wherein the floor is configured for supporting a live insect on the floor, and wherein the walls of the well are configured to inhibit the live insect from climbing out of the well; and
a second component comprising a post configured for placement within the well;
wherein the first component and the second component are threadably engageable to crush an insect between the post and the floor of the well.

16. The insect processing tool of claim 15, wherein the first component and the second component comprise handles for manual threaded engagement to crush the insect.

17. The insect processing tool of claim 15, wherein the first component comprises a liquid flow channel intersecting the well.

18. The insect processing tool of claim 17, wherein the liquid flow channel has an entry port configured for coupling to a syringe and an exit port configured for coupling to a sample reservoir.

19. A method of testing an insect for a vector transmitted infectious disease comprising:
in a non-laboratory environment, crushing the insect and mixing crushed portions of the insect with a liquid carrier;
in a non-laboratory environment, applying the liquid carrier that has been mixed with the crushed portions of the insect to a lateral flow assay strip.

20. The method of claim 19, comprising filtering the liquid carrier that has been mixed with the crushed portions of the insect before applying it to the lateral flow assay strip.
